(19) United States
(12) Patent Application Publication
(10) Pub. No.: US 2023/0197188 A1
(43) Pub. Date: Jun. 22, 2023

(54) SYSTEMS AND METHODS FOR MODIFYING THE FOLDING TRAJECTORY AND FACILITATING FOLDING OF POLYPEPTIDE CHAINS INTO NATIVE, NON-NATIVE, AND ARTIFICIAL CONFORMATIONS

(71) Applicant: Strenic LLC, Bethesda, MD (US)

(72) Inventor: Irina Nikolaevna Sorokina, Bethesda, MD (US)

(21) Appl. No.: 17/945,581

(22) Filed: Sep. 15, 2022

Related U.S. Application Data
(60) Provisional application No. 63/244,262, filed on Sep. 15, 2021.

(51) Int. Cl. G16B 15/20

(52) U.S. Cl. CPC G16B 15/20

(57) ABSTRACT

The present invention relates to peptide manipulation systems and methods for modifying the folding trajectory and facilitating the folding of a polypeptide chain into native, non-native, or artificial conformations. The invention comprises applying movement restriction(s) and/or directional rotation(s) to a plurality of locations along a peptide backbone. The applied movement restriction(s) and/or directional rotation(s) are sufficient to achieve twisting or other conformation changes of different portions of the peptide backbone and thereby modify peptide folding trajectory and facilitate peptide folding.

### CROSS-REFERENCE TO RELATED APPLICATIONS

This application claims the benefit of U.S. Provisional Application 63/244,262, filed Sep. 15, 2021, titled “SYSTEM AND PROCESS FOR FACILITATED FOLDING OF POLYPEPTIDE CHAINS,” which is herein incorporated by reference in its entirety.

### BACKGROUND

Proteins fold robustly and reproducibly in vivo, but many cannot fold in vitro in isolation from cellular components. The pathways to proteins' native conformations, either in vitro or in vivo, remain largely unknown. The most common accepted mechanism for protein folding in vivo is that folding is driven by thermodynamics, in particular solely by the decrease in Gibbs free energy. However, attempting to apply approaches based on the models of unassisted, spontaneous protein folding to simulate protein folding in vitro and in silico have not led to accurate, reliable and reproducible folding outcomes for majority of proteins or polypeptide chains.

There are currently no known systems and methods for facilitating and/or simulating protein folding via direct mechanical manipulations. Rather, as discussed above, existing models rely on the principle that folding will occur spontaneously without an external energy source aiding in the folding. Many approaches also rely on a fundamental assumption that the polypeptide backbone behaves as a freely jointed chain.

There is a need for an improved approach to facilitating protein folding in vitro and in silico that is able to augment folding trajectories and facilitate polypeptide chain folding into native, non-native and/or artificial stable folded conformations. There is also a need for an approach that addresses the issue that protein folding may not be a spontaneous external energy independent process, that external forces may play a role in protein folding, and that proteins may not behave as freely jointed chains during the folding process.

### SUMMARY

The present invention overcomes these limitations by creating a protein folding system and method which relies on direct manipulation of the peptide backbone in vitro and in silico in order to model and/or facilitate in vivo and/or in vitro protein folding. The invention provides improved protein folding simulations and in vitro folding protocols capable of achieving realistic protein folding pathways by applying external forces such as restraining and rotating forces to a polypeptide chain which can be applied in a way that allows manipulating polypeptide backbones such that all or at least portions of the polypeptide backbone can be prevented from behaving as a freely jointed chain.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a system for facilitated folding of polypeptide chains in accordance with an exemplary embodiment of the invention.

FIG. 2 illustrates an exemplary process for facilitated folding of polypeptide chains in accordance with an exemplary embodiment of the present invention.

FIG. 4 illustrates one embodiment of the computing architecture that supports an embodiment of the inventive disclosure.

FIG. 5 illustrates components of a system architecture that supports an embodiment of the inventive disclosure.

FIG. 6 illustrates components of a computing device that supports an embodiment of the inventive disclosure.

FIG. 7 illustrates components of a computing device that supports an embodiment of the inventive disclosure.

FIG. 8 illustrates exemplary peptides and their corresponding characteristics used for molecular dynamics simulations according to one embodiment of the invention.

FIG. 9 illustrates a schematic representation of an energy-dependent peptide folding protocol according to an embodiment of the invention.

FIG. 10 illustrates folding rates of exemplary peptides in association with varying molecular dynamics simulations according to one embodiment of the invention.

FIG. 11 illustrates exemplary folding characteristics of exemplary peptides in association with different directional rotations of the polypeptide backbone according to one embodiment of the invention.

FIG. 12 illustrates exemplary folding characteristics of exemplary peptides in association with applied movement restriction(s) and/or directional rotation(s) according to one embodiment of the invention.

FIG. 13 illustrates exemplary folding characteristics of exemplary peptides in association with applied movement restriction(s) and/or directional rotation(s) according to one embodiment of the invention.

FIG. 14 illustrates exemplary folding characteristics of exemplary peptides in association with applied movement restriction(s) and/or directional rotation(s) according to one embodiment of the invention.

### DETAILED DESCRIPTION

The inventive system and method (hereinafter sometimes referred to more simply as “system” or “method”) described herein significantly improves protein folding outcomes by manipulating polypeptide chains via direct manipulation. Specifically, the inventive system applies restraining and/or rotating forces at various locations along a polypeptide backbone to create at least one of twisting or other change of conformation of a polypeptide chain, performs various iterations of twisting or other change of conformation over time and provides a controlled release of the polypeptide chain to allow folding into native, non-native, and/or artificial conformations. The inventive system described herein provides an approach that incorporates new factors into the protein folding mechanism which have not previously been implemented into protein folding simulations or in vitro protein folding protocols.

One or more different embodiments may be described in the present application. The instant invention may be implemented with each embodiment individually or any combination of the embodiments disclosed. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, physical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process which may be applied to in silico or in vitro protein manipulations. However, in vitro applications may include solely manual manipulations according to the process descriptions or block in figures, computer implemented or assisted manipulations via computer executable instructions (e.g. instructions for controlling a robotic manipulator) or combinations of manual and computer implemented manipulations. Alternate implementations are included within the scope of various embodiments in which, for example, functions may be executed out of order from that shown or discussed, including substantially concurrently or in reverse order, depending on the functionality involved, as would be understood by those having ordinary skill in the art.

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

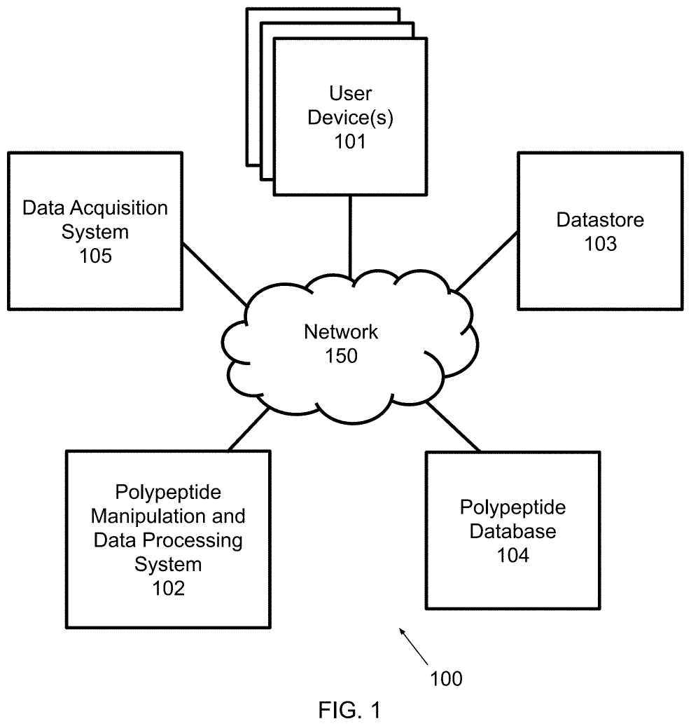

FIG. 1 illustrates an exemplary embodiment of a polypeptide manipulation system 100 according to one embodiment. The system includes user device(s) 101, a polypeptide manipulation and data processing system 102, a datastore 103, data acquisition system 105, polypeptide database 104, and a network 150 over which the various systems communicate and interact. The various components described herein are exemplary and for illustration purposes only and any combination or subcombination of the various components may be used as would be apparent to one of ordinary skill in the art. Other systems, interfaces, modules, engines, databases, and the like, may be used, as would be readily understood by a person of ordinary skill in the art, without departing from the scope of the invention. Any system, interface, module, engine, database, and the like may be divided into a plurality of such elements for achieving the same function without departing from the scope of the invention. Any system, interface, module, engine, database, and the like may be combined or consolidated into fewer of such elements for achieving the same function without departing from the scope of the invention. All functions of the components discussed herein may be initiated manually or may be automatically initiated when the criteria necessary to trigger action have been met.

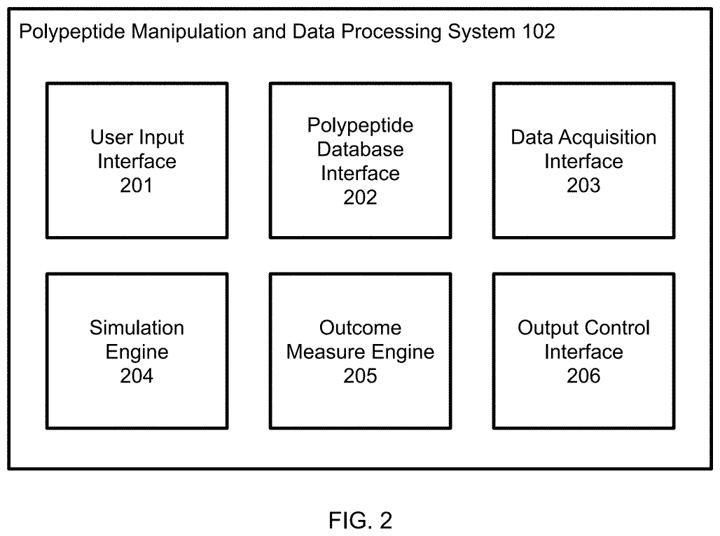

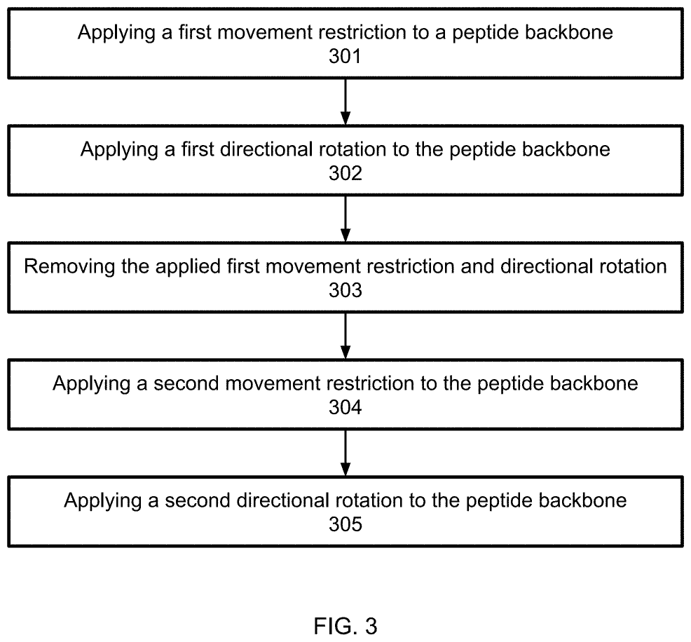

User device(s) 101 may be used to communicate with a polypeptide manipulation and data processing system 102 and a datastore 103 via network 150. User device(s) 101 may function to allow a user to specify or select one or more polypeptide chains to be manipulated. In one embodiment, this may involve using the user device(s) 101 to enter one or more polypeptide sequences and transmitting such sequences to the polypeptide manipulation and data processing system 102. In one embodiment, the user device(s) 101 may be used to select one or more polypeptide chains from a list of polypeptide chains stored in a datastore 103 or stored in the polypeptide manipulation and data processing system 102. The list of polypeptide chains may include common natural protein sequences, and/or artificial sequences, and/or smaller polypeptide chains such as polypeptide chains which commonly make up larger polypeptide chains. User device(s) 101 may also be used to communicate settings and preferences for the polypeptide manipulation such as target intermediate or final conformations resulting from polypeptide manipulation, manipulation parameters such as forces and other parameters such as those discussed below with respect to FIG. 2 to be used in the polypeptide manipulation. In one embodiment, the user device(s) 101 may communicate custom settings and parameters. In one embodiment, the user device(s) 101 may be used to select settings and parameters from lists of settings and parameters stored in a datastore 103 or stored in the polypeptide manipulation and data processing system 102.

The polypeptide manipulation and data processing system 102 manipulates one or more polypeptide sequences based on a set of settings and parameters. These settings and parameters may be preset settings and parameters defined within the polypeptide manipulation and data processing system 102, settings and parameters received from user device(s) 101, and/or settings and parameters received from a datastore 103. The polypeptide manipulation and data processing system 102 may also receive an input specifying one or more polypeptide chains to be manipulated. This may involve receiving one or more particular polypeptide sequences from user device(s) 101 in the form of custom sequences and/or selection of one or more polypeptide sequences from a list as described above. In one embodiment, the polypeptide manipulation and data processing system 102 generates a polypeptide chain based on received input such as a received amino acid sequence.

In one embodiment, the polypeptide manipulation and data processing system 102 may be a server or cloud-based software application for performing polypeptide manipulation in silico. In one embodiment the polypeptide manipulation and data processing system 102 may take the form of a local application installed and running on a user device 101. In one embodiment, the polypeptide manipulation and data processing system 102 may include a variety of components for manipulating a polypeptide in vitro. Such components may include manipulators for applying mechanical forces, electrical forces, and/or magnetic forces. Some exemplary components may include one or more robotic arms and end effectors, components such as various solid phases for covalent, non-covalent and/or affinity protein immobilization, molecular tweezers, lasers, nanodevices, microfluidic devices, electromagnetic field generators, and the like which may be used separately or as one or more end effectors on robotic arms.

The datastore 103 may store manipulation settings and parameters used for a given polypeptide manipulation. The datastore 103 may store outcomes of manipulations such as resulting conformations, and videos of the manipulation and conformation changes. For example, the applied forces and their timing among other parameters such as, but not limited to, those discussed under FIG. 2 below may be stored in datastore 103. In addition or alternative, the outcome of a particular manipulation may also be stored such as a video of the resulting polypeptide manipulation and resulting folding. In one embodiment, the datastore 103 may also be integrated into the user device 101 or polypeptide manipulation and data processing system 102. The datastore may also store lists of peptide chains that can be manipulated or used to build larger polypeptide chains to be manipulated. The datastore 103 may also store lists of settings and parameters that a user can choose from in order to perform polypeptide chain manipulations. This may involve preset lists of common manipulation settings and parameters or may include custom made lists of presets. In one embodiment, the datastore 103 may store settings and parameters for simulating polypeptide manipulation in silico. In one embodiment, the datastore 103 may store settings and parameters for performing an in vitro polypeptide manipulation, for example settings and parameters to control a robotic device and end effectors, and/or settings and parameters to monitor changes in the conformation of a polypeptide in real time, in order to manipulate a polypeptide chain. The datastore 103 may also store real-time data collected to monitor the changes in the conformation of a polypeptide folding in vitro. Examples of such real-time monitoring include, but are not limited to, fluorescence, circular dichroism, and nuclear magnetic resonance spectra.

Polypeptide database 104 may comprise data associated with a plurality of polypeptide and/or protein structures, folding conformations, and/or folding trajectories. The polypeptide database 104 may comprise data associated with a plurality of known proteins and/or polypeptide chains. The polypeptide database 104 may be updated over time as new data associated with proteins and/or polypeptides becomes available, including but not limited to naturally occurring proteins, artificial proteins, misfolded protein conformations, and the like. In one aspect, data from the polypeptide database 104 is provided to polypeptide manipulation and data processing system 102 for use in computing outcome measures related to peptide backbone manipulation as described in more detail below.

Client device(s) 101 include, generally, a computer or computing device including functionality for communicating (e.g., remotely) over a network 150. Data may be collected from client devices 101, and data requests may be initiated from each client device 101. Client device(s) 101 may be a server, a desktop computer, a laptop computer, personal digital assistant (PDA), an in- or out-of-car navigation system, a smart phone or other cellular or mobile phone, or mobile gaming device, among other suitable computing devices. Client devices 101 may execute one or more client applications, such as a web browser (e.g., Microsoft Windows Internet Explorer, Mozilla Firefox, Apple Safari, Google Chrome, and Opera, etc.), or a dedicated application to submit user data, or to make prediction queries over a network 150.

In particular embodiments, each user device 101 may be an electronic device including hardware, software, or embedded logic components or a combination of two or more such components and capable of carrying out the appropriate functions implemented or supported by the user device 101. For example and without limitation, a user device 101 may be a desktop computer system, a notebook computer system, a netbook computer system, a handheld electronic device, or a mobile telephone. The present disclosure contemplates any user device 101. A user device 101 may enable a network user at the user device 101 to access network 150. A user device 101 may enable its user to communicate with other users at other user devices 101.

A user device 101 may have a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user device 101 may enable a user to enter a Uniform Resource Locator (URL) or other address directing the web browser to a server, and the web browser may generate a Hyper Text Transfer Protocol (HTTP) request and communicate the HTTP request to server. The server may accept the HTTP request and communicate to the user device 101 one or more Hyper Text Markup Language (HTML) files responsive to the HTTP request. The user device 101 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device 101 may also include an application that is loaded onto the user device 101. The application obtains data from the network 150 and displays it to the user within the application 533 interface.

Exemplary user devices are illustrated in some of the subsequent figures provided herein. This disclosure contemplates any suitable number of user devices, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing system may perform at different times or at different locations one or more steps of one or more methods described or illustrated herein, where appropriate.

Network cloud 150 generally represents a network or collection of networks (such as the Internet or a corporate intranet, or a combination of both) over which the various components illustrated in FIG. 1 (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 421 or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to clients devices or other devices in response to HTTP or other requests from clients devices or other devices. A mail server is generally capable of providing electronic mail services to various clients devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiments, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the invention.

FIG. 2 illustrates an exemplary embodiment of the polypeptide manipulation and data processing system 102. The system 102 comprises user input interface 201, simulation engine 204, output control interface 206, data acquisition interface 203, polypeptide database interface 202, and outcome measure engine 205. The various components described herein are exemplary and for illustration purposes only and any combination or subcombination of the various components may be used as would be apparent to one of ordinary skill in the art. Other systems, interfaces, modules, engines, databases, and the like, may be used, as would be readily understood by a person of ordinary skill in the art, without departing from the scope of the invention. Any system, interface, module, engine, database, and the like may be divided into a plurality of such elements for achieving the same function without departing from the scope of the invention. Any system, interface, module, engine, database, and the like may be combined or consolidated into fewer of such elements for achieving the same function without departing from the scope of the invention. All functions of the components discussed herein may be initiated manually or may be automatically initiated when the criteria necessary to trigger action have been met. In one aspect, the system 102 of FIG. 2 is operable to employ the exemplary process described in association with FIG. 3.

User input interface 201 is operable to obtain user input associated with peptide manipulation. In one aspect user input comprises a set of parameters/settings to be used in peptide manipulation. Exemplary user input may comprise an indication of and/or settings or parameters associated with at least one movement restriction and/or at least one directional rotation to be applied to a peptide backbone. In one aspect, user input comprises at least one of a force to be applied (including, but not limited to, at least one of force type (e.g. mechanical, electrical, magnetic, etc.), force constant(s) and force magnitude to be used), a rate of directional rotation to be applied, a direction of rotation to be applied (e.g. clockwise or counter clockwise about the longitudinal axis of the peptide backbone), a duration of application of at least one of movement restriction(s) and directional rotation(s), and one or more locations at which each manipulation will be applied. Additional settings/parameters may be specified or determined from user input, such as those discussed below in association with FIG. 3, among others as would be apparent to one of ordinary skill in the art.

Simulation engine 204 is operable to execute computer simulations in order to apply movement restriction(s) and/or directional rotation(s) associated with the obtained user input. In one aspect, simulation engine 204 performs molecular dynamics simulations in association with the settings/parameters established based on the user input. Simulation engine 204 may execute a plurality of simulation processes associated with applying at least one of movement restriction(s) and/or directional rotation(s), such as that described in FIG. 3 below. In one aspect, the applied movement restriction(s) and/or directional rotation(s) implemented via simulation engine 204 augment the folding trajectory and facilitate the folding of a peptide chain into at least one of a native, non-native, and artificial conformation.

Output control interface 206 is operable to output control signals to physical peptide manipulation components. In one aspect, the control signals are provided to components configured for performing in vitro peptide manipulation. Exemplary components include, but are not limited to one or more robotic arms and end effectors, components such as various solid phases for covalent, non-covalent and/or affinity protein immobilization, molecular tweezers, lasers, optical tweezers, magnetic tweezers, magnetic beads, electromagnets, piezoelectric devices, piezoacoustic devices, nanodevices, microfluidic devices, electromagnetic field generators, and the like.

Data acquisition interface 203 is operable to capture data associated with peptide manipulation. In one aspect, data acquisition interface 203 is operable to obtain data from an external system or component which acquires data associated with in vitro peptide manipulation. In one aspect, data acquisition interface 203 is operable to obtain, alone, or in combination/cooperation with simulation engine 204, snapshots or screenshots of peptide manipulation throughout the manipulation process. In one aspect, data acquisition interface obtains peptide manipulation data at least one of before, during, and after the application and/or removal of movement restriction(s) and/or directional rotation(s) applied to the peptide.

Polypeptide database interface 202 is operable to at least one of obtain information from a polypeptide database and provide information to a polypeptide database. In one aspect, polypeptide database interface 202 obtains information associated with known and/or expected folding trajectories and/or conformations associated with known peptide sequences (e.g. known proteins). In one aspect, polypeptide database interface 202 obtains information associated with known and/or expected folding trajectories and/or conformations associated with artificial peptide sequences (e.g. artificial proteins). In one aspect, polypeptide database interface 202 obtains information associated with rates or folding durations associated with at least one of known and unknown peptide chains.

Outcome measure engine 205 is operable to compute at least one outcome measure based on data obtained in association with at least one of data acquisition interface 203 and polypeptide database interface 202. In one aspect, outcome measure engine is operable to compute at least one of a folding trajectory of the polypeptide chain, a resulting folded conformation, a folding accuracy, the number or proportion of the native contacts formed by the amino acids, and a time to achieve the resulting folded conformation. In one aspect, the outcome measure engine 205 computes at least one of a measure of similarity and a measure of difference between at least one of the computed folding trajectory as compared to an expected folding trajectory and the computed resulting folded conformation as compared to an expected resulting folded conformation. In one aspect, the outcome measure engine 205 computes at least one of a measure of similarity and a measure of difference between at least one of the computed folding trajectory as compared to an expected folding trajectory and the computed resulting folded conformation as compared to an expected resulting folded conformation. In one aspect, the outcome measure engine 205 computes at least one of a measure of similarity and a measure of difference between an observed folding trajectory and an expected folding trajectory. In one aspect, the comparison of computed or observed folding trajectory and an expected folding trajectory can be used to identify if and when a peptide chain achieves or progresses towards a mis-folded conformation. In one aspect, the outcome measure engine 205 computes at least one of a measure of similarity and a measure of difference between at least one of a computed folding time and an expected folding time in order to determine a degree of augmentation resulting from the applied peptide manipulations.

FIG. 3 illustrates an exemplary embodiment of a process for manipulating polypeptide chains to facilitate folding of the polypeptide chain into a native, non-native or artificial stable folded conformation. The process comprises applying a first movement restriction to a peptide backbone 301, applying a first directional rotation to the peptide backbone 302, removing the applied first movement restriction and directional rotation 303, applying a second movement restriction to the peptide backbone 304, and applying a second directional rotation to the peptide backbone 305. The process may comprise additional steps, fewer steps, and/or a different order of steps without departing from the scope of the invention as would be apparent to one of ordinary skill in the art.

At step 301, the process comprises applying at least one first movement restriction to a peptide backbone 301. The movement restriction may comprise applying at least one of a force and a torque to the peptide backbone. In one aspect, applying at least one first movement restriction may comprise applying a plurality of movement restrictions at one or more locations along the peptide backbone. The movement restriction may comprise a restriction to at least one of prevent rotation of the peptide backbone and limit rotation of the peptide backbone. In one aspect, limiting rotation may comprise limiting at least one of an amount of rotation and a rate of rotation of the peptide backbone. In one aspect, movement restriction(s) is/are applied via at least one of an established force field, electrical force(s), magnetic force(s), mechanical force(s), covalent and/or non-covalent binding forces or characteristics, and other force(s). In one aspect, applying at least one first movement restriction may comprise applying at least one force to an amino acid, where the at least one force is of sufficient magnitude to restrict movement of at least one of the amino acid and portions of the peptide backbone in proximity to the amino acid. In one aspect, applying at least one first movement restriction may comprise applying at least one force to a plurality of amino acids, where the at least one force is of sufficient magnitude to restrict movement of at least the plurality of amino acid and portions of the peptide backbone in proximity to the plurality of amino acids. In one aspect, the movement restriction(s) are applied to at least one of an N-terminal amino acid, a C-terminal amino acid, and any other amino acid of the peptide backbone. In one aspect, applying movement restriction(s) comprises modifying at least one of a temperature, a chemical composition, and a viscosity of a solvent in which the peptide is located.

At step 302, the process comprises applying at least one first directional rotation to the peptide backbone. In one aspect, the direction of rotation comprises at least one of clockwise or counterclockwise rotation about the longitudinal axis of the peptide backbone. The directional rotation may comprise applying at least one of a force and a torque to the peptide backbone. In one aspect, applying at least one first directional rotation may comprise applying a plurality of directional rotations at one or more locations along the peptide backbone. In one aspect, directional rotation(s) is/are applied via at least one of an established force field, electrical force(s), magnetic force(s), mechanical force(s), covalent and/or non-covalent binding forces or characteristics, and other force(s). In one aspect, applying at least one first directional rotation may comprise applying at least one force to an amino acid, where the at least one force is of sufficient magnitude to rotate at least one of the amino acid and portions of the peptide backbone in proximity to the amino acid. In one aspect, applying at least one first directional rotation may comprise applying at least one force to a plurality of amino acids, where the at least one force is of sufficient magnitude to rotate at least the plurality of amino acid and portions of the peptide backbone in proximity to the plurality of amino acids. In one aspect, the directional rotation(s) are applied to at least one of an N-terminal amino acid, a C-terminal amino acid, and any other amino acid of the peptide backbone. In one aspect, the directional rotation(s) is/are applied using an enforced rotation method. In one aspect, the directional rotation(s) is/are applied over a threshold time period. In one aspect, the directional rotation(s) is/are applied using a flexible axis approach with a defined reference rotation rate.

At step 303, the process comprises removing the applied first movement restriction(s) and directional rotation(s). In one aspect, removing the restrictions and/or rotations may involve removing all applied manipulations simultaneously. In one aspect, removing may involve removing the applied manipulations in a particular order. In one aspect, removing may comprise at least one of removing individual applied manipulations one at a time, removing groups of applied manipulations one at a time. In one aspect, removing the applied movement restriction(s) and directional rotation(s) comprises a controlled release of the constraints on the peptide backbone, such as a gradual or stepwise decrease in the forces applied in order to impose the restrictions and/or rotations. In one aspect, the controlled release may involve removing all or some applied manipulations with additional application of manipulations at one or more of the same locations as previously applied forces and/or additional application of forces at one or more different locations as previously applied constraints. In one embodiment, the controlled release may involve maintaining one or more applied manipulation at the end of this step.

At step 304, the process comprises applying at least one second movement restriction to the peptide backbone. This may be performed in the same fashion as applying the first movement restriction as described in step 301 above. The second movement restriction(s) may be applied at the same and/or different locations as the at least one first movement restriction(s) applied in step 301.

At step 305, the process comprises applying at least one second directional rotation to the peptide backbone. This may be performed in the same fashion as applying the first directional rotation as described in step 302 above. The second directional rotation(s) may be applied at the same and/or different locations as the at least one first directional rotation(s) applied in step 302.

In one aspect, the combined application of movement restriction(s) and directional rotation(s) are performed in such a way as to induce at least one of a twisting of the peptide backbone and a change of conformation of the peptide backbone. In one aspect, the twisting or other change of conformation of the peptide backbone serves to modify the folding trajectory and facilitate the folding of a polypeptide chain into native, non-native, and/or artificial conformations. In one aspect, the application of movement restriction(s) and directional rotation(s) augment peptide folding by accelerating folding by a defined factor (e.g. reduce the time needed to achieve a folded, stable conformation). In one aspect, the application of movement restriction(s) and/or directional rotation(s) are adjusted and/or modified as necessary in order to achieve a desired or predefined twisting or other conformation change of the peptide backbone.

In one aspect, the above application of movement restriction(s) and directional rotation(s) may comprise performing the applications in silico as part of a computer simulation. For example, molecular dynamics simulation software may be used to apply the movement restriction(s) and directional rotation(s). In one aspect, the computer simulations are performed using at least one of a constant temperature, a constant pressure, and a defined force constant. In one aspect, the movement restriction(s) and directional rotation(s) are applied in vitro. In one aspect, in vitro application of movement restriction(s) and directional rotation(s) are performed using at least one of optical tweezers, magnetic tweezers, magnetic beads, electromagnets, piezoelectric devices, and piezoacoustic devices. In one aspect, the process is used to produce at least one artificial protein having at least one of a native and a non-native folding conformation.

In one aspect, the process may further comprise capturing images before, during, and/or after at least one of the application and removal of movement restriction(s) and directional rotation(s). In one aspect, capturing images comprises obtaining snapshots or screenshots via a computer simulation based application and removal of the movement restriction(s) and directional rotation(s).

In one aspect, the process may further comprise computing at least one outcome measure associated with peptide manipulation. In one aspect, the outcome measure(s) are associated with at least one of folding trajectory, resulting folded conformation, folding accuracy, the number or proportion of the native contacts, and a time to achieve the resulting folding conformation. In one aspect, the folding accuracy comprises at least one of a measure of similarity and a measure of difference between at least one of the computed folding trajectory as compared to an expected folding trajectory and the computed resulting folded conformation as compared to an expected resulting folded conformation. In one aspect, the process may further comprise identifying at least one of at least one native folding confirmation achieved, at least one non-native folding conformation achieved, at least one stable folding conformation achieved, at least one non-stable folding conformation achieved, at least one functional conformation achieved, at least one intermediary confirmation achieved (e.g. an alpha helix), and at least one misfolded conformation achieved.

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

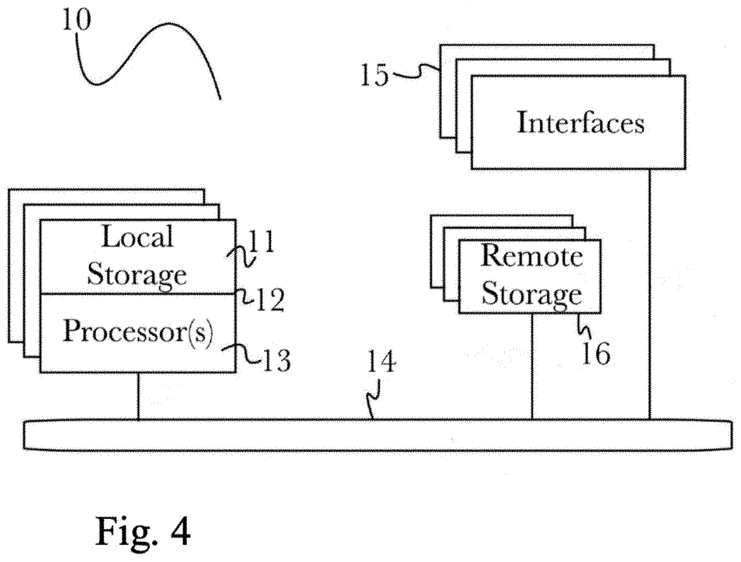

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments). Any of the above mentioned systems, units, modules, engines, controllers, components or the like may be and/or comprise hardware and/or software as described herein. For example, the polypeptide manipulation and data processing system 102 and subcomponents thereof may be and/or comprise computing hardware and/or software as described herein in association with FIGS. 4-7. Furthermore, any of the above mentioned systems, units, modules, engines, controllers, components, interfaces or the like may use and/or comprise an application programming interface (API) for communicating with other systems units, modules, engines, controllers, components, interfaces or the like for obtaining and/or providing data or information.

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

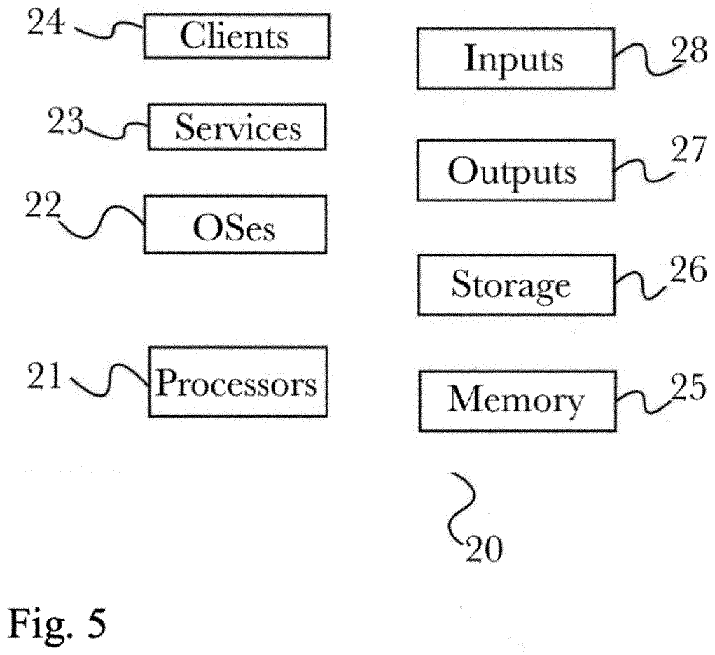

In some embodiments, systems may be implemented on a standalone computing system. Referring now to FIG. 5, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 4). Examples of storage devices 26 include flash memory, magnetic hard drive, CD-ROM, and/or the like.

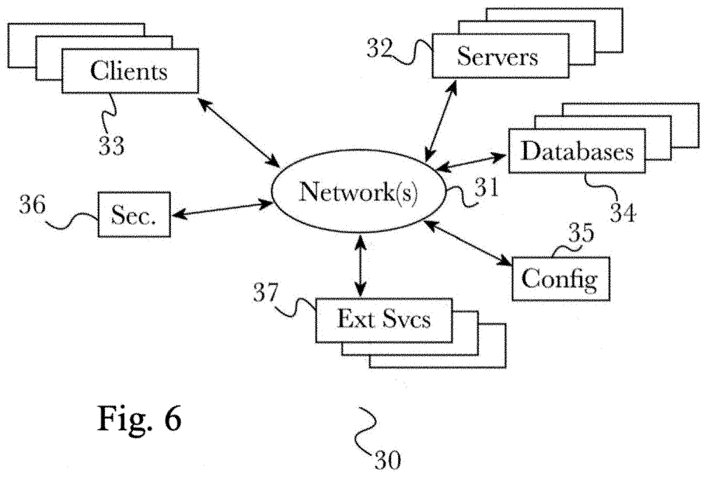

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise's or user's premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

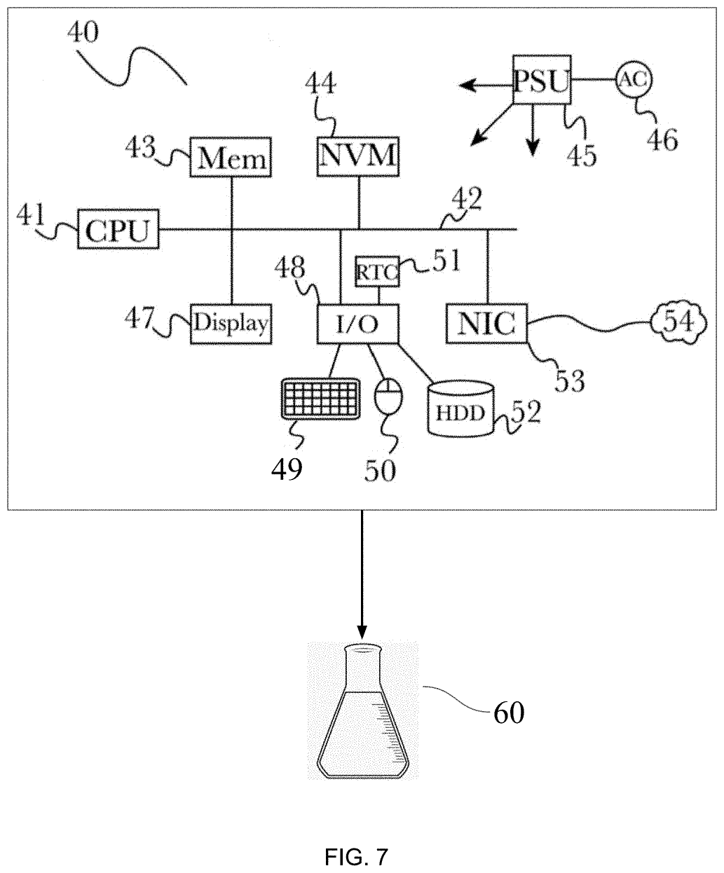

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices). The computing system 40 or output from the computing system may be applied to performing in vitro peptide manipulation and protein formation such as in a laboratory setting as represented by reference numeral 60. In one aspect, the computer system 40 may be used to at least partially control in vitro manipulations. In one aspect, the computer system 40 may supplement another computer system in performing in vitro manipulations. In one aspect, the computer system 40 may provide output (e.g. simulation properties/results) for use by a second computer system wherein the second computer system performs in vitro manipulations based at least partially on the output provided by computer system 40. In one aspect, in vitro manipulations may be performed by mechanical manipulation using at least a portion of computer simulation information and/or output from computer system 40.

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

A first example implementation and corresponding results of the above inventive concepts is included below.

Once they are synthesized in a living cell, the majority of proteins rapidly attain their distinctive biologically active three-dimensional structures, called native conformations. These conformations are robustly achieved in vivo via a folding process that involves interactions of the folding chain with molecular chaperones and other maturation factors. The native conformations of many proteins can be predicted using modern artificial intelligence methods, but the folding process leading to those native conformations usually cannot be reproduced either in silico or in vitro, except for those of a few relatively short polypeptides. Knowledge of the intermediates in the folding pathways and the mechanisms that enable them is essential for determining the points of intervention at which folding and misfolding processes can be altered.

The current dominant model of protein folding was prompted by early observations that some small proteins are able to fold in vitro into their native conformations spontaneously, in isolation from other proteins or cellular components. These observations gave rise to the thermodynamic hypothesis of protein folding, which in turn led to the development of the physical model that describes protein folding as a thermodynamically favorable, unassisted process. In a more recent, refined form, this model includes the description of a rugged funnel-shaped energy landscape, in which the various unfolded, unstructured conformations occupy the high-free-energy brim of the funnel. As the polypeptide chains fold, they sample conformations with progressively decreasing Gibbs free energy until they reach the native conformation, which is presumed to occupy the global thermodynamic minimum at the bottom of the funnel. The sampling of conformations during the folding process is assumed to occur via random thermal motions. The driving force of protein folding is assumed to be the decrease in free energy to the global minimum.

In summary, the current general physical model of protein folding describes a process that occurs in a closed system in the absence of external sources of energy. It assumes that folding starts from a random, unstructured conformation and proceeds unassisted, with no apparent requirement for the folding chain to interact with other proteins or macromolecular cellular components.

As the first step in exploring the feasibility of a protein folding machine capable of facilitating the attainment of native structure by mechanical manipulation of the peptide backbone, we performed molecular dynamics simulations augmented by application of torsion to the peptide backbones. During the simulations, the C-termini of various polypeptides were mechanically rotated either clockwise or counterclockwise, while the motions of their N-termini were restricted. We compared the trajectories of both types of simulations with the folding of the same peptides without the application of torque. In our experiments, directional rotation of the C-terminal amino acids with simultaneous limitation of the movements of the N-termini indeed facilitated the formation of native structures in five diverse alpha-helical peptides.

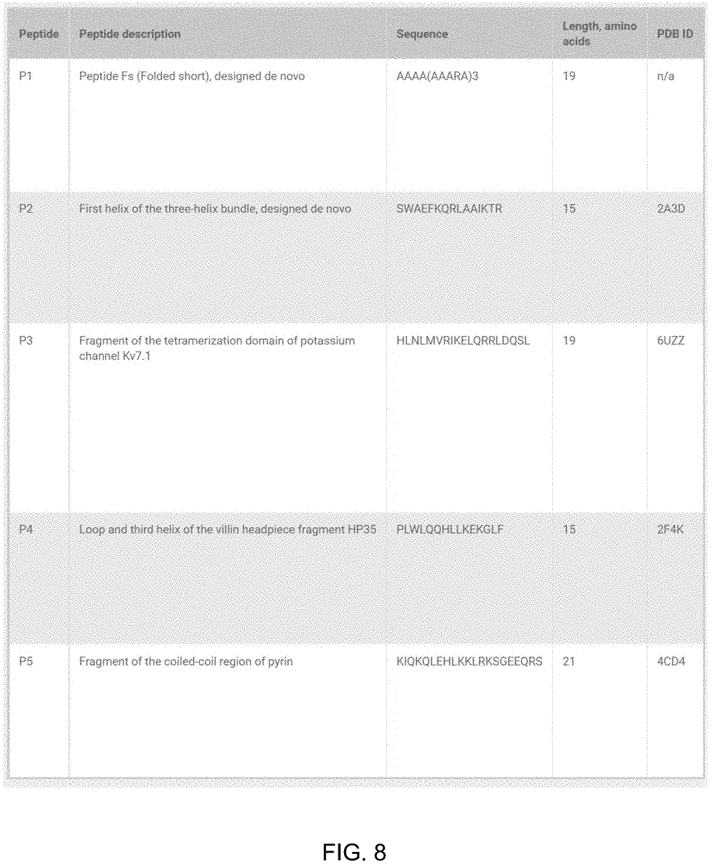

The initial stretched structures of peptides (shown in FIG. 8) were generated using ICM software. We aligned a peptide along the X-axis and solvated it in a dodecahedron box in the case of the simulations of unassisted folding and triclinic box in all other cases, with minimum distance of 1.5 nm between a peptide and the simulations box. Potassium and sodium ions were added to neutralize the charges in the system. The system was then minimized with the steepest descent algorithm, equilibrated for 100 ps in the NVT ensemble using V-rescale thermostat for temperature coupling, and continued in the NPT ensembles for 1 ns using V-rescale thermostat and Berendsen barostat. After the equilibration, we kept temperature and pressure constant at 300 K and 1 bar respectively, using Nose-Hoover thermostat and isotropic Parinello-Rahman barostat.

For all simulations, we used the ff14SB force field with the TIP3P water model and ion parameters modified by Joung and Cheatham. Electrostatic interactions were calculated using particle-mesh Ewald (PME) summation with a Fourier grid spacing of 0.135 nm. For non-bonded Coulomb and Lennard-Jones interactions, 1 nm cutoff was used. We constrained the hydrogen bonds with the LINCS algorithm and used a 2-fs integration time step.

To exert an external mechanical torque to the C-termini of the peptides, we applied the enforced rotation. To this end, we restrained the positions of the O and N atoms of the C-terminal alanine to keep it aligned with the X-axis, about which the rotation was applied. The restraints with a force constant of 10000 kJ/mol*nm² were applied only for the YZ-plane, so the C-terminal amino acid could move along the X-axis. In addition, we restrained the O atom of the C-terminal amino acid in the X direction with a force constant of 5 kJ/mol*nm² and N and Ca atoms of the N-terminal alanine with a force constant 10000 kJ/mol*nm² in all directions. The C-terminal amino acid was rotated using a flexible axis approach (Vflex2) with a reference rotation rate of 60 degrees/ps and a force constant of 1500 kJ/mol*nm².

The GROMACS package version 2020.236 was used for all simulations and trajectory analyses. The simulations were carried out on CUDA-enabled GPUs with Turing architecture, running Ubuntu 18.04. For visualization of protein structures and trajectories, the programs ICM-Pro 3.924 and VMD 1.9.337 were used.

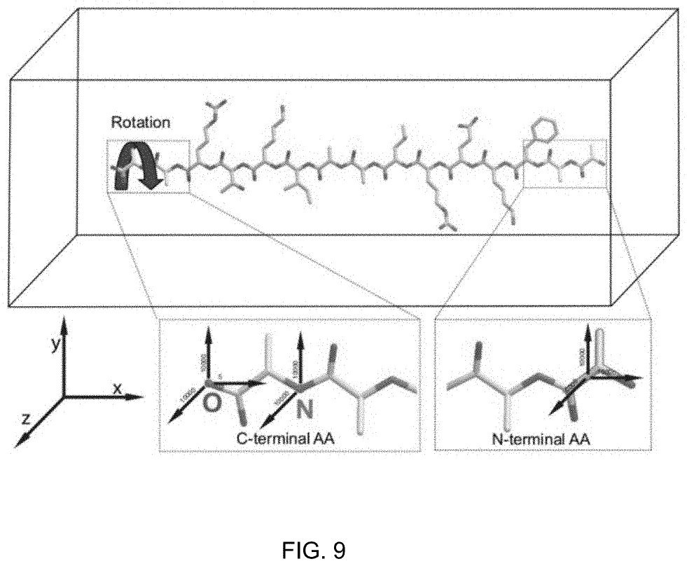

FIG. 9 illustrates a schematic representation of the energy-dependent peptide folding protocol employed in this study. The force vectors applied to the C- and N-termini of a peptide in the simulation box are shown by arrows. All force values are in kJ/mol*nm². The curled arrow indicates the direction of the clockwise rotation of the peptides that resulted in the accelerated productive folding of all peptides to their helical conformations. The restrained groups are shown by green outline. We compared the folding trajectories of the peptide to which a mechanical force was applied to rotate the C-terminal amino acid in one of the two possible directions—either clockwise as in FIG. 9, or counterclockwise—with the trajectories for the same peptide which was allowed to fold without any motion restriction or application of any mechanical force (referred to as “unassisted folding” below). As an additional control, we ran a fourth type of simulation, where motion restraints were applied to both ends of each peptide but the torque was omitted. Each of the four types of simulations were repeated three times, giving 12 simulations for each peptide.

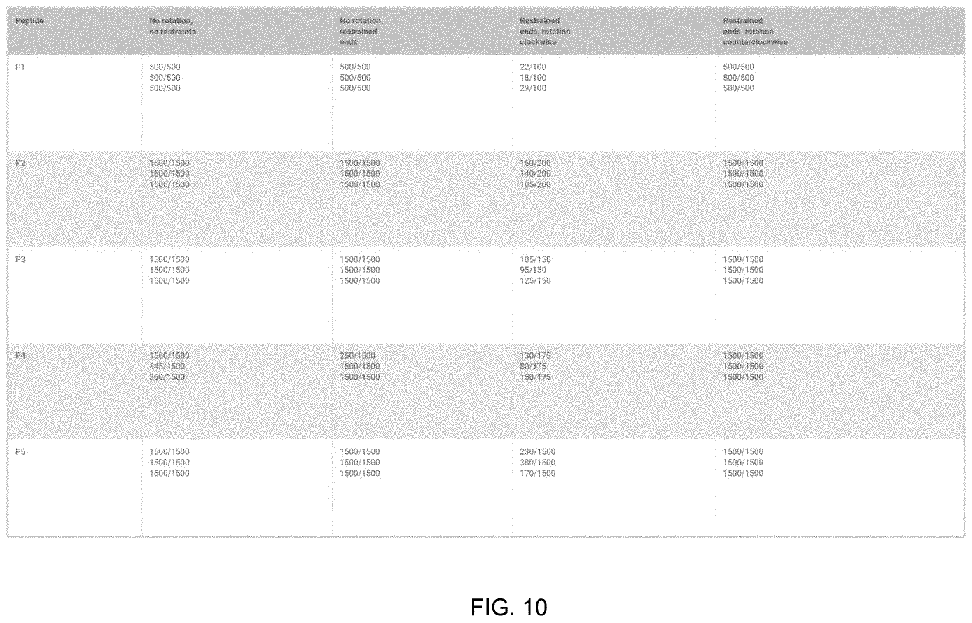

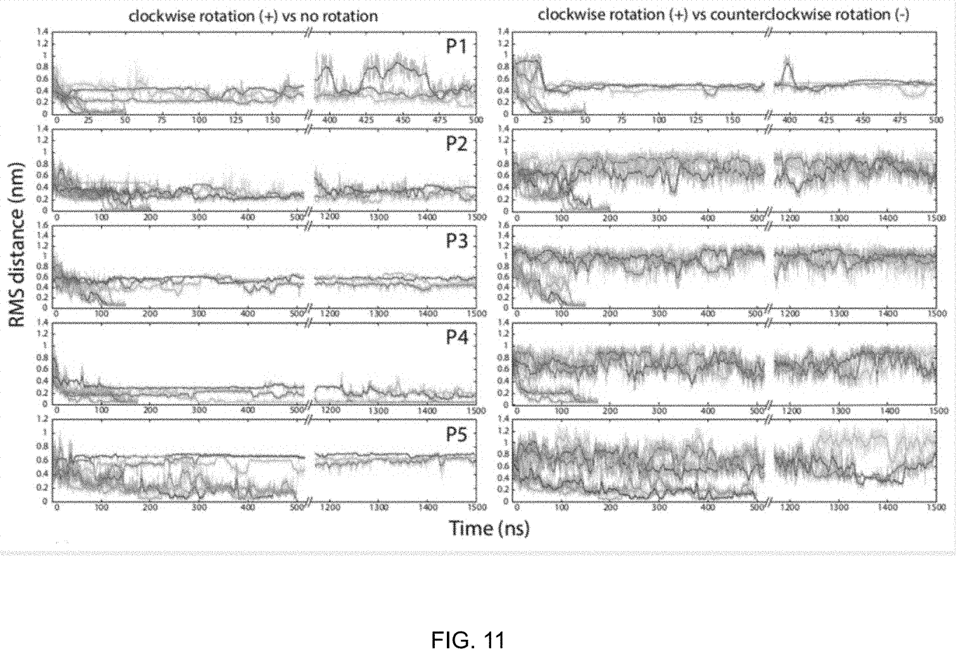

The experiments were run on five peptides that are known to adopt α-helical conformations in their folded form (FIG. 8). Two of these have been designed de novo, and the other three are parts of naturally occurring proteins. The folding of the peptides was monitored by calculating the root mean square deviation (RMSD) distance of the peptide backbone from the native structure of the same fragment determined by X-ray crystallography (peptides P2-P5), or computed ab initio (peptide P1). The results of the simulations for each peptide when folded unassisted in the standard force field, and when an external torque force was added to the field, are presented in FIG. 10 and FIG. 11. Within our simulation lengths, we observed the completion of unassisted folding into the native-like α-helical structure only in some runs for one peptide, P4, which represents the third helix and preceding loop in the villin headpiece domain HP35. Other peptides remained essentially unfolded throughout the 500-1500-nanosecond runs. The peptides also failed to fold when their ends were restricted in mobility but torque was not applied (FIG. 10). In contrast, when the external torsion force was applied to the C-termini of the peptides in the clockwise direction, as described in Methods and illustrated in FIG. 1, peptides P1-P4 all folded into α-helical structures and were brought within 0.2 nm RMSD from their native structures in every run, typically within the first 100-200 ns of simulation. These peptides stayed in the native or nearly-native conformations for the remainder of the experiments. Peptide P5 was a special case; similarly to P1-P4, it adopted a compact conformation early in the experiments, but remained only partially folded for the duration of all runs (FIG. 11).

For all five peptides, folding was observed when the rotation force was applied to the C-terminal amino acid in the clockwise direction (FIG. 9). In contrast, the torque applied to the C-terminus counterclockwise with the same force constant did not facilitate folding of P1-P3 and P5, and may have inhibited folding of P4 (FIG. 11).

Thus, the directional rotation of the C-terminal amino acid with simultaneous restriction of the movements of the N-terminal amino acid facilitated the formation of native structures in five diverse α-helical peptides, confirming that such constraints can have significant consequences for folding dynamics. Strikingly, application of mechanical force accelerated the folding of P4, a fragment of an on-pathway folding intermediate of the well-studied villin headpiece domain HP35, which is one of the fastest-folding protein domains known. The several-fold increase in the rate of P4 folding that was achieved in our experiments seems to suggest that the postulated “physical limit of folding” of HP35 as a whole could be overcome by a protein folding machine. The other four peptides in our experiments likewise attained their α-helical structure in the presence of the rotating force, but did not reach their native conformations when allowed to fold unassisted, even though we ran the control unassisted simulations for ˜10 times longer than the simulations that included the application of the external force (FIG. 10). Some of those peptides might take a very long time to reach their native conformations without application of an external force, whereas others might never fold unassisted, if their unfolded states are more stable than the folded conformations.

A second example implementation and corresponding results of the above inventive concepts is included below.

We next conducted all-atom molecular dynamics simulations of three small protein domains, whose folding from an extended state was augmented by the application of rotational force to the C-terminal amino acid, while the movement of the N-terminal amino acid was restrained. The simulation protocol was changed to apply the backbone rotation and movement restriction for a limited time at the start of simulation. As will be shown below, this transient application of a mechanical force to the peptide is sufficient to accelerate, by at least an order of magnitude, the folding of three protein domains with different structure to their native or nearly-native structures. Our in silico experiments show that the native or native-like fold may be attained more readily when the movements of the polypeptide are biased by external forces and constraints.

In one aspect backbone twisting (or other conformation change) was applied for a defined time at the start of the simulation, and then the remainder of the simulation continued without restraints. The force was applied transiently for the first 250 ns of simulation, with the reference rate 0.36 degree/ps and a force constant 10000 kJ mol-1 nm-2. As in earlier experiments, we applied the rotation against the direction of an α-helix. Control simulations were performed in the boxes with mostly the same properties as in the rotation-augmented runs, but without restraints or manipulations of the polypeptide at any point. To monitor the protein folding process, we used several metrics throughout the simulation, collecting snapshots every 100 ps. At the whole-domain level, we measured the RMSD distance from the known native three-dimensional structure of the folded domain and the fraction of native contacts formed by all amino acids within the domain. At the residue level, we tested whether the residue was incorporated into a correct element of the secondary structure and whether it formed a native contact.

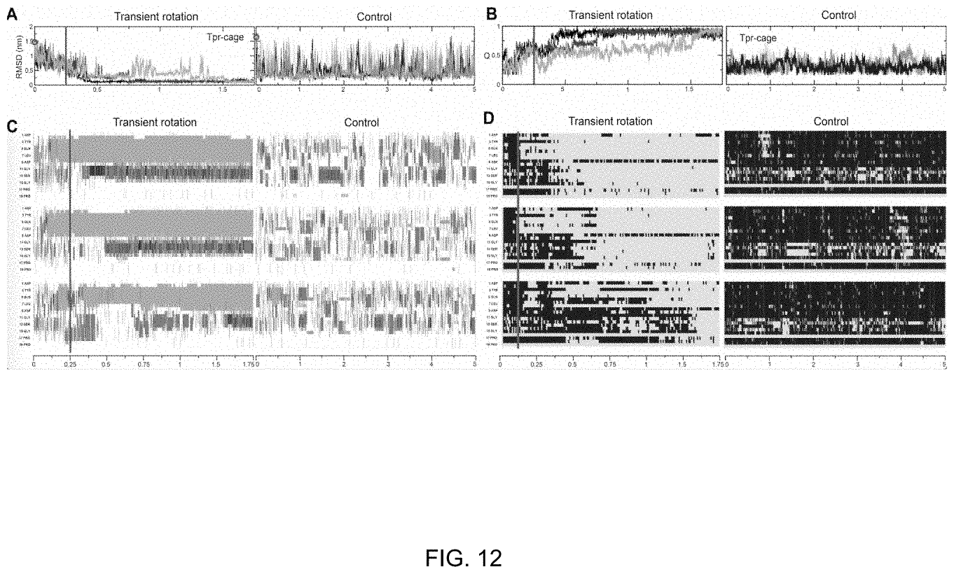

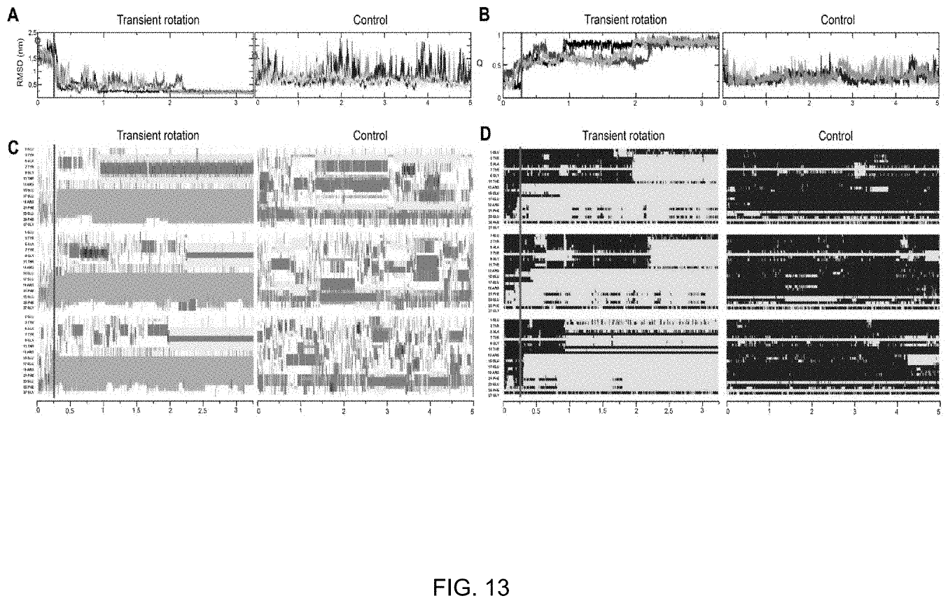

For our simulations, we selected protein domains whose folding in vitro and in molecular dynamics simulations has been extensively studied. Two of those domains are proteins designed for rapid folding and stability. They are Tpr-cage domain (PDB ID 2jof, 20 amino acids), which consists of a helix-coil-turn motif and a short proline tail that packs against the helix, and BBA domain (PDB ID 1fme, 28 amino acids), a derivative of a Zn finger that is stable in a metal-free form, consisting of two β-strands followed by an α-helix. Both peptides were placed in their simulation boxes in the stretched conformations, and both peptides rapidly folded to their native structures in the presence of directional rotation. The details of the folding process are presented in FIGS. 12 and 13.

The molecular dynamics (MD) simulations were carried out using the GROMACS-2020.4 package with CHARMM36m force field (July 2020 release). The stretched structures of proteins were produced with the ICM-Pro program. The proteins were aligned along the X-axis and solvated in triclinic boxes with TIP3P water and potassium and chloride ions. The system was then minimized with the steepest descent algorithm and equilibrated with NVT/NPT ensembles for 1 ns at each equilibration cycle. The MD simulations were performed at a constant temperature of 300 K with the v-rescale thermostat and a time constant of 0.1 ps. The pressure was coupled at 1 bar with Parrinello-Rahman barostat using a time constant of 2 ps. Electrostatic forces were calculated using the particle-mesh Ewald method with a Fourier grid spacing of 0.16 nm. Short-range Van der Waals interactions were force-switched off from 0.8 to 1.2 nm. The hydrogen-containing bonds were constrained using the LINCS algorithm, and 2 fs integration time step was used. Snapshots were collected every 100 ps.

The rotation of C-terminal amino acid was performed using the enforced rotation method implemented in Gromacs as in our previous study. The rotation was applied during the first 250 ns to the C-terminal amino acid using the flexible axis approach (Vflex2) with reference rotation rate 0.36 degree/ps (1 rotation per 1 ns) and force constant 10000 kJ*mol-1*nm-2, and continued without restraints and without applied rotation thereafter. Most control simulations were performed in the same box and with the same parameters, but without any initial restraint or rotation; see text for more discussion of some control runs for Trp cage and BBA domains.

The RMS distances from the database-deposited native structure were calculated in Gromacs. Acquisition of secondary structure and the fraction of native contacts were assessed using VMD software. VMD was also used for trajectory visualization. Native contacts for an amino acid were considered to be formed if that amino acid has the same contacts as in the reference X-ray structure within a cutoff 8 Å. The fraction of native contacts (Q) was calculated as Q=N/Nall where N is the number of the native contacts at a given time and Nall is the number of contacts in the reference X-ray structure.

A more detailed view of the folding process is provided by the analysis of secondary structure and native contacts at the residue level. Trp-cage domain and BBA domain each contain a helical segment in their native structure—respectively, in the N-terminal and C-terminal region of the polypeptide chain. As can be seen in FIGS. 12 (panel C) and 13 (panel C), these helices rapidly formed at the initial, constrained stage of the simulation, when the backbone twist was applied. After the switch to unassisted simulation, the helices remained stable. The other parts of these domains, i.e, the C-terminal portion of Trp-cage and the N-terminal portion of the BBA domain, did not form helices during the backbone rotation stage, and rapidly acquired their native conformations after the release from the constrained rotation stage, so that the entire native structures were formed and packed correctly.

In the unassisted control simulations, the two polypeptides formed neither helices nor other elements of the secondary or tertiary structure. Short substructures with helix-like or strand-like properties could be seen, but these states were occupied only transiently and/or were located in the regions that have different type of structure in the native form of the protein (FIG. 12, C, and FIG. 13, C).

For each target, the summaries of independent steered simulations can be compared side by side at the residue level, providing valuable visual information about the folding pathways (FIG. 12, D, and FIG. 13, D). It is evident that for the Trp-cage domain, the later stages of simulation are qualitatively very similar in all three runs, with the same residues forming the native contacts in the same order; the main differences are in the times between the end of the steered rotation phase and the beginning of the productive folding event, and the rate of folding (FIG. 12, D). The same is true of the BBA domain; here again, the order in which specific residues formed their native contacts was nearly the same each time, and the difference was mostly in the time shift in the entire sequence of events (FIG. 13, D). Thus, these two protein molecules did not explore alternative folding trajectories in our experiments; even if such multiple folding pathways exist, our steered simulation approach appears to direct the folding process into a specific pathway for each domain.

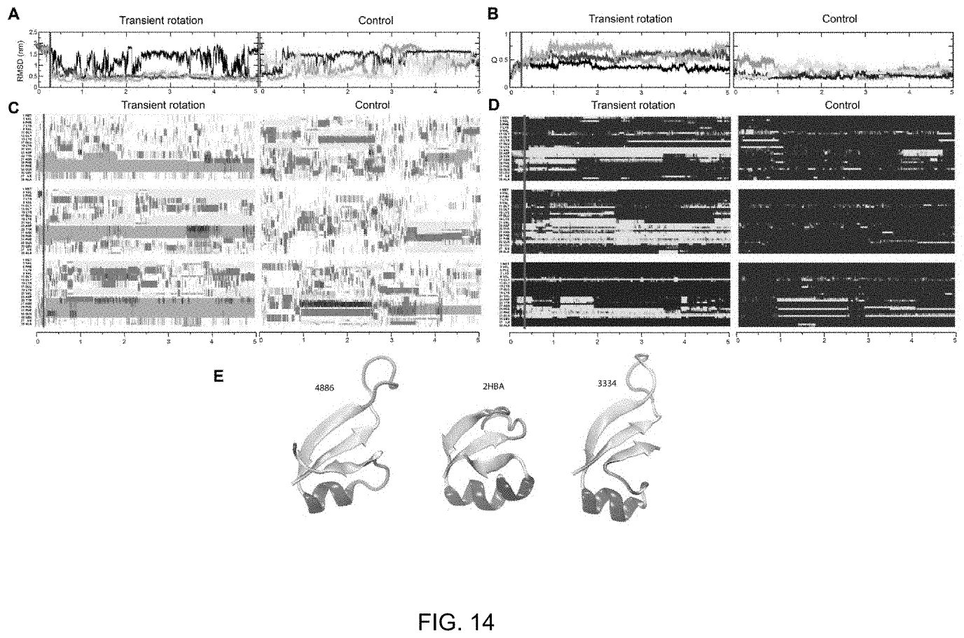

We next studied folding of a naturally occurring protein domain that is somewhat longer than Trp-cage and BBA domains. The fragment of the N-terminal domain of ribosomal protein L9 from Geobacillus stearothermophilus (NTL9-39; PDB ID 2hba, 39 amino acids, K 12M mutant), consists of a β-sheet formed by three anti-parallel β-strands, and an α-helix that is located between strands 2 and 3 along the peptide chain and is packed against one edge of the sheet. The folding of the 39-residue form, which does not include the long α-helix connecting the N-terminal and C-terminal domains of the L9 protein, has been studied in vitro and in silico before. We simulated folding of this protein in the same way as for the other two domains, except that the simulation box was 10.5 nm long in the X axis direction, and therefore the starting peptide was not fully stretched (calculated fully-stretched length 12.8 nm). The results of our folding simulations for this domain are shown in FIG. 14.

In the rotation-assisted simulations, NTL 9 folded into stable and compact form quickly, within 1-1.5 μs (FIG. 14, panels A and B). This is approximately 20-fold faster than 29 μs folding rate reported for the same domain in the literature. The main folding intermediate, in which amino acids 22-35 formed a helix, was observed in all three runs while the transient rotation was still applied. After the restraints and external forces were removed, in two runs the C-terminus of the helix (amino acids 30-35) relaxed into a loop while the rest of the helix was retained, the β-strands were formed elsewhere in the molecule, and NTL9 attained compact conformation. Without assistance, NTL9 did not fold for the duration of the simulations FIG. 14, A and B, right half-panels).

The structure that was formed in the rotation-assisted runs remained stable for the entire simulation time, but displayed interesting local differences from the known native conformation of NTL9. Focusing on the run 2, the majority of the fold, i.e., the helix and strands 1 and 2 that directly interact with it, were all formed nearly perfectly. The third beta β-strand was also observed, completing the three-stranded β-sheet similar to the wild-type fold. The examination of the snapshots in the course of the simulation, however, revealed a dynamic transition between two distinct conformations. One conformation was nearly identical to the native structure (FIG. 14, E, left), but it existed only for the total of 0.5 μs throughout the rotation-assisted simulation. For a much longer period, 1.2 μs, the direction of the third strand within the fold was reversed from antiparallel to parallel, which required the connecting loop to stretch over the edge of the folded domain, frazzling the C-terminus of the α-helix (FIG. 14, E, right).

The results of the above applied manipulations indicate this novel approach to peptide manipulation successfully modified folding trajectories of proteins. In one aspect, folding was accelerated approximately ten fold or more as compared to what is known in the art. The inventive approach also provides a means for detecting misfolding and the characteristics associated with misfolding occurrences and/or detection of folding trajectory inflection points where folding towards non-native, but stable conformations occurs.

### ADDITIONAL CONSIDERATIONS

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and Bis false (or not present), A is false (or not present) and Bis true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for creating an interactive message through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A system for modifying the folding trajectory and facilitating the folding of a polypeptide chain into native, non-native, or artificial conformations, the system comprising:
at least one computing processor; and
memory comprising instructions that, when executed by the at least one computing processor, enable the computing system to:
apply at least one first movement restriction preventing rotation of at least one first portion of a peptide backbone;
apply at least one first directional rotation to at least one second portion of the peptide backbone, wherein the first directional rotation and first movement restrictions are applied simultaneously;
remove the applied first movement restriction and first directional rotation;
apply at least one second movement restriction preventing rotation of at least one third portion of a peptide backbone; and
apply at least one second directional rotation to at least one fourth portion of the peptide backbone, wherein the second directional rotation and second movement restriction are applied simultaneously.

2. The system of claim 1, wherein the applied movement restrictions and directional rotation cause at least one of twisting of the peptide backbone and a change in conformation of the polypeptide chain.

3. The system of claim 1, wherein applying the at least one first movement restriction comprises applying at least one of a force and a torque at a first location along the peptide backbone.

4. The system of claim 1, wherein applying the at least one first directional rotation comprises applying at least one of a force and a torque at a second location along the peptide backbone.

5. The system of claim 1, wherein the applying directional rotations and movement restrictions are performed in silico as part of a computer simulation.

6. The system of claim 1, wherein the applying directional rotations and movement restrictions are performed in vitro.

7. The system of claim 1, wherein the applying directional rotations comprises augmenting an established force field with the addition of a mechanical rotational force.

8. The system of claim 1, wherein the directional rotations are applied to at least one of an N-terminal amino acid, a C-terminal amino acid, and any other amino acid of the peptide backbone.

9. The system of claim 1, wherein the movement restrictions are applied to at least one of an N-terminal amino acid, a C-terminal amino acid, and any other amino acid of the peptide backbone.

10. The system of claim 1, wherein the applying directional rotations comprises applying at least one of electrical forces, magnetic forces, mechanical forces, and other forces.

11. The system of claim 1, wherein the applying movement restrictions comprises applying at least one of covalent and non-covalent binding characteristics to at least one of an amino acid within the peptide backbone, an atom within the peptide backbone, an atom in a peptide side chain, and an atom within an entity tightly bound to the peptide.

12. The system of claim 1, wherein the applying movement restrictions comprises modifying at least one of a temperature, a chemical composition, and a viscosity of a solvent in which the peptide is located.

13. A system for modifying the folding trajectory and facilitating the folding of a polypeptide chain into native, non-native, or artificial conformations, the system comprising:
at least one peptide manipulator configured to physically manipulate the polypeptide chain, wherein the peptide manipulator is configured to:
apply at least one first movement restriction preventing at least one of rotation or other movement of at least one first portion of a peptide backbone;
apply at least one first directional rotation or other movement to at least one second portion of the peptide backbone, wherein the first directional rotation or other movement and first movement restrictions are applied simultaneously;
remove the applied first movement restriction and first directional rotation or other movement;
apply at least one second movement restriction preventing rotation or other movement of at least one third portion of a peptide backbone; and
apply at least one second directional rotation or other movement to at least one fourth portion of the peptide backbone, wherein the second directional rotation or other movement and second movement restriction are applied simultaneously.

14. The system of claim 13, wherein the polypeptide manipulator is configured to apply movement restrictions and directional rotations or other movements using at least one of optical tweezers, magnetic tweezers, magnetic beads, electromagnets, piezoelectric devices, and piezoacoustic devices.

15. A method for modifying the folding trajectory and facilitating the folding of a polypeptide chain into native, non-native, or artificial conformations, the method comprising:
applying at least one first movement restriction preventing rotation or other movement of at least one first portion of a peptide backbone;
applying at least one first directional rotation to at least one second portion of the peptide backbone, wherein the first directional rotation or other movement and first movement restrictions are applied simultaneously;
removing the applied first movement restriction and first directional rotation or other movement;
applying at least one second movement restriction preventing rotation or other movement of at least one third portion of a peptide backbone; and
applying at least one second directional rotation to at least one fourth portion of the peptide backbone, wherein the second directional rotation or other movement and second movement restriction are applied simultaneously.
